(19) United States
(12) Patent Application Publication
(10) Pub. No.: US 2020/0390961 A1
(43) Pub. Date: Dec. 17, 2020

(54) ANTI-REFLUX LEAKPROOF ENEMATOR

(71) Applicant: Ningbo Albert Novosino Co., Ltd, Ningbo (CN)

(72) Inventors: Yonggui Zhang, Ningbo (CN); Haibo Hu, Ningbo (CN)

(21) Appl. No.: 16/571,605

(22) Filed: Sep. 16, 2019

(30) Foreign Application Priority Data
Jun. 13, 2019 (CN) 201910512324.6
Jun. 13, 2019 (CN) 201920889416.1

(51) Int. Cl. A61M 3/02

(52) U.S. Cl. CPC A61M 3/0262; A61M 3/0216; A61M 3/0279

(57) ABSTRACT

Disclosed are various embodiments for an anti-reflux enemator that is leakproof and easy-to-clean. Generally, the anti-reflux enemator includes a nozzle, an enema bulb fluidly coupled to the nozzle, the enema bulb being configured to store solution therein and, in response to a squeezing force applied to the enema bulb, direct the solution through the nozzle and a nozzle outlet, an anti-reflux coupler positioned between the enema bulb and the nozzle through which the solution passes from the enema bulb to the nozzle, the anti-reflux coupler comprising an anti-reverse diaphragm configured to prevent reflux of the solution into the enema bulb from the nozzle, and at least one one-way air valve positioned relative to an aperture located at a base of the enema bulb.

### CROSS-REFERENCE TO RELATED APPLICATIONS

This application claims the benefit of and priority to Chinese Patent Application No. 201920889416.1, filed Jun. 13, 2019 and Chinese Patent Application No. 201910512324.6, filed Jun. 13, 2019, the contents of which being incorporated by reference in their entireties herein.

### TECHNICAL FIELD

This invention belongs to the technical field of enemators and, more specifically, describes an anti-reflux, leakproof, and easy-to-clean enemator.

### BACKGROUND

Enemators include syringe-type devices that can be utilized by patients and medical practitioners for cleansing the body, such as vaginal, anal, and other bodily cavities. Generally, enemators are filled with solution, such as clean or soapy water, which is injected into a cavity of a person by hand squeezing a bulb or similar apparatus. During this process, the solution may “reflux,” where some solution is returned into the enemator, thereby contaminating the solution and affecting the use of conventional enemators. Moreover, many types of enemators do not prevent leakage of solution. For instance, due to poor design and manufacturing, existing enemators in the art are unable to store solution for a long period of time without leakage. To address this, some enemators use a fixed leakproof structure so that they are not easy to clean, have a limited length of use, and a high manufacturing and purchasing cost. Also, a joint line on the side of the connected syringe nozzle exists in some enemators to ease manufacturing costs; however, the joint line often scratches the human body during use. Due to poor manufacturing and design, the outlet of the syringe nozzle is easily blocked, leading to a poor user experience.

### BRIEF SUMMARY OF INVENTION

Disclosed are various embodiments for an anti-reflux enemator that is leakproof and easy-to-clean. Generally, the anti-reflux enemator includes three components that are independently removable from one another, which facilitates cleaning, while providing strong sealing mechanisms that prevent leakage. In some embodiments, the three components of the anti-reflux enemator include a nozzle, an enema bulb fluidly coupled to the nozzle, and an anti-reflux coupler positioned between the enema bulb and the nozzle through which the solution passes from the enema bulb to the nozzle. The enema bulb is configured to store solution therein and, in response to a squeezing force applied to the enema bulb, direct the solution through the nozzle and out from a nozzle outlet. The anti-reflux coupler includes an anti-reverse diaphragm configured to prevent reflux of the solution into the enema bulb from the nozzle. In some embodiments, the anti-reflux enemator includes one or more one-way air valves positioned relative to an aperture located at a base of the enema bulb.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the disclosure. Moreover, in the drawings, like reference numerals designate corresponding parts throughout the several views.

FIG. 1 is a schematic cross-sectional view showing the structure of the invention device connected to a large nozzle (e.g., a large syringe pipe).

FIG. 2 is a schematic cross-sectional view showing the structure of the invention device connected to a small nozzle (e.g., a small syringe pipe).

FIG. 3 is a cross-sectional view of callout region A-A in FIG. 1.

FIG. 4 is a cross-sectional view of callout region B-B in FIG. 1.

FIG. 5 is a partially enlarged schematic view of callout region A in FIG. 1.

FIG. 6 is a partially enlarged schematic view of callout region B in FIG. 1.

FIG. 7 is a schematic view showing the state of water outflow of anti-reflux plug in the invention device.

FIG. 8 is a schematic view showing structure of the first external thread connector in the device of the invention.

### DETAILED DESCRIPTION

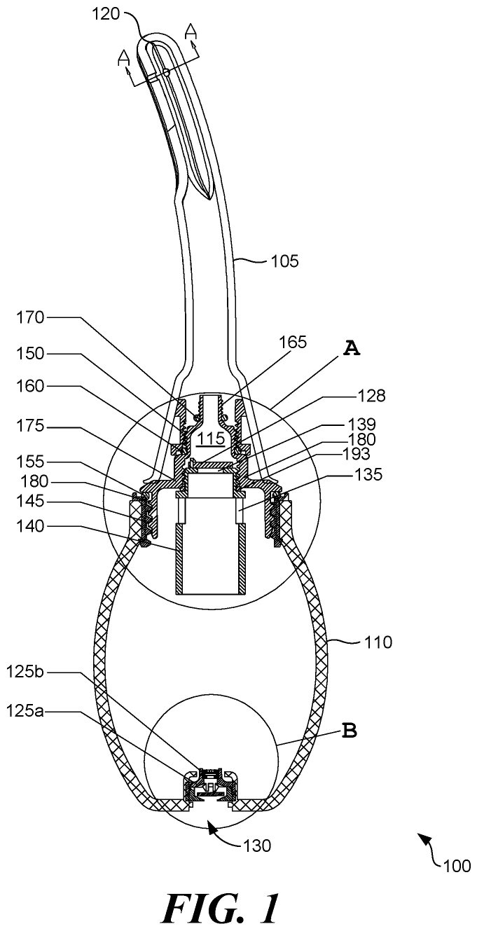

The present disclosure generally relates to an anti-reflux enemator that is leak resistant and easy to assemble and clean. Referring now collectively to FIGS. 1-8, an anti-reflux enemator 100 is shown according to various embodiments. Generally, the anti-reflux enemator 100 can include three components that are independently removable from one another, which facilitates quick cleaning while providing strong sealing mechanisms that prevent leakage. In some embodiments, the three components of the anti-reflux enemator include a nozzle 105, an enema bulb 110 fluidly coupled to the nozzle 105, and an anti-reflux coupler 115 fluidly coupled to each of the nozzle 105 and the enema bulb 110. However, it understood that, in alternative embodiments, the anti-reflux enemator 100 is not limited to three components.

In some embodiments, the anti-reflux coupler 115 is positioned between the enema bulb 110 and the nozzle 105. For instance, in some embodiments, a first portion (e.g., approximately a first half) of the anti-reflux coupler 115 is nested with an interior of the enema bulb 110, while a second portion (e.g., approximately a second half) of the anti-reflux coupler 115 is nested within an interior of the nozzle 105.

The enema bulb 110 is configured to store solution therein and, in response to a squeezing force applied to the enema bulb 110, direct the solution through the nozzle 105 and out through one or more nozzle outlets 120 positioned on or near a top, distal end of the nozzle 105. To this end, a squeezing motion can cause solution or other content expelled from the nozzle 105, which can be inserted into a bodily cavity for cleaning or other medical purpose.

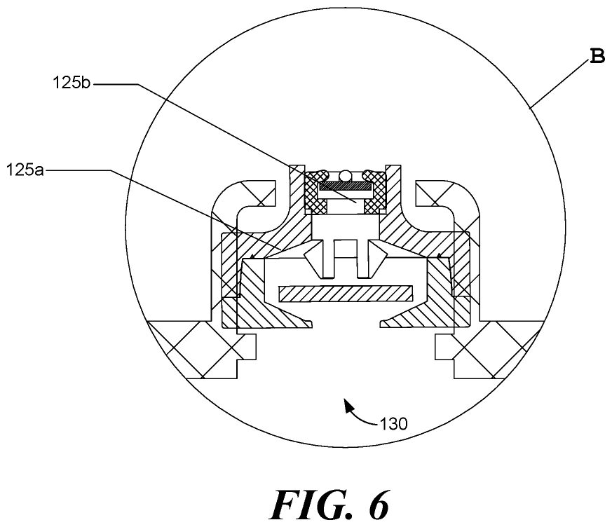

Further, in some embodiments, the anti-reflux enemator 100 can include at least one one-way air valve 125a, 125b positioned relative to an aperture 130 located at a base of the enema bulb 110. For instance, in some embodiments, the anti-reflux enemator 100 can include a first one-way air valve 125a and a second one-way air valve 125b positioned relative to the aperture 130, where the aperture 130 is located at a base of the enema bulb 110. The at least one air valve 125 positioned at the base of the anti-reflux enemator 100 permits the intake of air, thereby facilitating the flow of solution and preventing the enema bulb 110 from being deflated after being squeezed. FIG. 6 illustrates an enlarged view of callout region B, shown in FIG. 1.

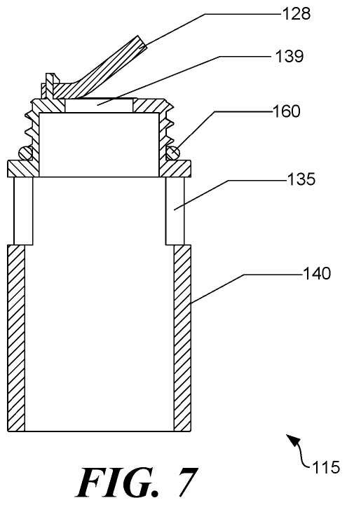

The anti-reflux coupler 115 includes an anti-reflux diaphragm 128 configured to prevent reflux of the solution into the enema bulb 110 from the nozzle 105. In some embodiments, the anti-reflux coupler 115 includes one or more inlets 135 for receiving at least a portion of the solution from an interior of the enema bulb 110, an outlet 139 for expelling the solution into the nozzle 105, and a check valve body 140. In some embodiments, the inlet 135 is positioned on the check valve body 140. The check valve body 140 can include a circular body extending into the interior of the enema bulb 110. FIG. 7 illustrates an enlarged view of callout region A, shown in FIG. 1.

Further, in some embodiments, the anti-reflux enemator 100 includes a first threaded connector 145 for detachably attaching the anti-reflux coupler 115 to the enema bulb 110 and a second threaded connector 150 for detachably attaching the nozzle 105 to the anti-reflux coupler 115. As such, it is understood that the anti-reflux coupler 115 directly couples the nozzle 105 to the enema bulb 110, although other suitable connection mechanisms can be employed.

The anti-reflux enemator 100 can further include a first sealing ring 155 for sealing the anti-reflux coupler 115 and the enema bulb 110 at the first threaded connector 145. For instance, the first sealing ring 155 prohibits solution, air, or other matter from traversing through a seam of the anti-reflux enemator 100. Additionally, the anti-reflux enemator 100 can include a second sealing ring 160 for sealing the nozzle 105 and the anti-reflux coupler 115. Like the first sealing ring 155, the second sealing ring 160 prohibits solution, air, or other matter from leaking through a seam of the anti-reflux enemator 100.

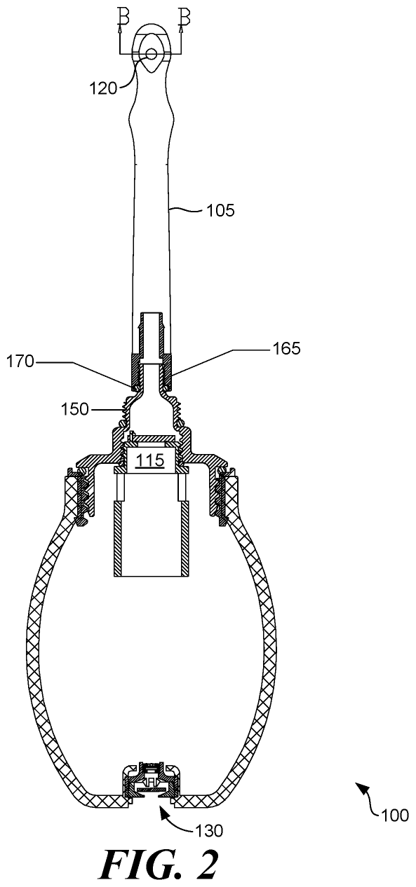

In further embodiments, the anti-reflux enemator 100 includes a third threaded connector 165 for detachably attaching the anti-reflux coupler 115 to a nozzle 105 having a different size and/or shape. For instance, as shown in FIG. 1, the nozzle 105 of the anti-reflux enemator 100 is a first nozzle 105 having a first predetermined length and a first predetermined diameter. In contrast, as shown in FIG. 2, the anti-reflux enemator 100 includes a second nozzle 105 having a second predetermined length and a second predetermined diameter different from the first predetermined length and the first predetermined diameter, respectively, where the second nozzle 105 of FIG. 2 is configured to replace or be used in place of the first nozzle 105 of FIG. 1. Notably, the nozzle 105 of FIG. 2 is shorter than the nozzle 105 of FIG. 1, which may be more ideal for certain uses. Additionally, in some embodiments, the diameter and the shape between the first nozzle 105 and the second nozzle 105 can be different, which can provide the user with more choices.

As shown in FIG. 1, the nozzle 105 is coupled to the anti-reflux coupler 115 via the second threaded connection 150, whereas, in FIG. 2, the nozzle 105 is coupled to the anti-reflux coupler 115 via the third threaded connector 165 positioned on the anti-reflux coupler 115. The anti-reflux enemator 100 can further include a third sealing ring 170 for sealing the anti-reflux coupler 115 and the nozzle 105 at the third threaded connector 165. The third sealing ring 170 can be positioned above the second sealing ring 160. In addition to the foregoing, in some embodiments, the anti-reflux enemator 100 can include a fourth threaded connector 175 configured to couple the check valve body 140 to a corresponding threaded connector 180 on a body of the anti-reflux coupler 115.

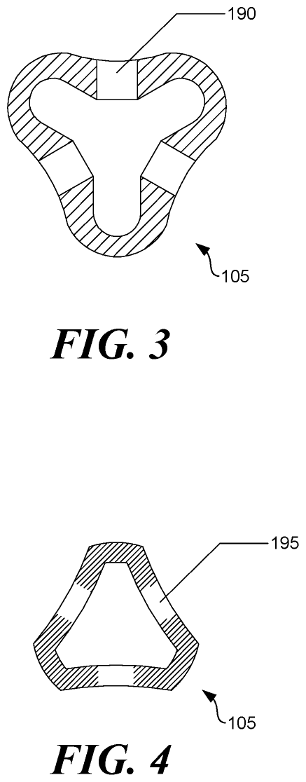

The difference in the shapes, diameters, and other characteristics of the different nozzles 105 are illustrated in the cross-sectional views of the nozzle 105 shown in FIGS. 3 and 4. For instance, FIG. 3 illustrates the cross-sectional view of the nozzle 105 of FIG. 1, while FIG. 4 illustrates the cross-sectional view of the nozzle 105 of FIG. 2. Generally, as shown in FIGS. 3 and 4, the nozzle 105 can include a triangular-shaped cross-section having a plurality of rounded corners.

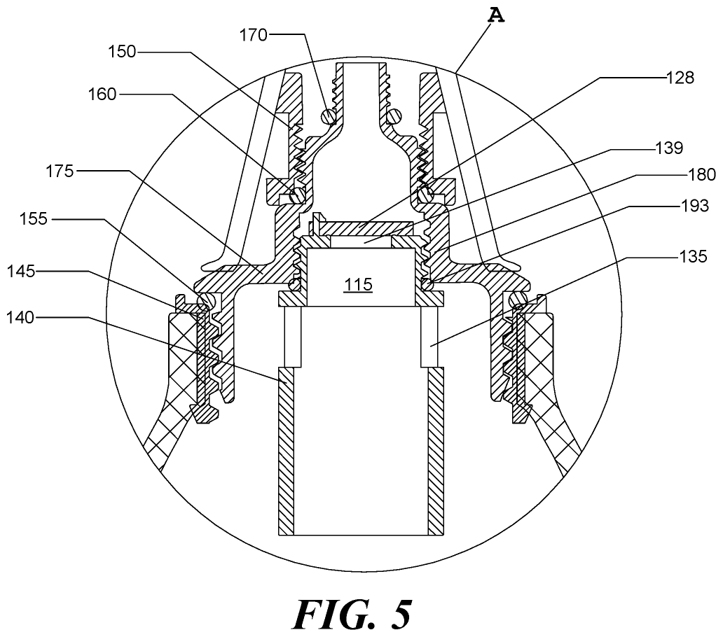

Referring next to FIG. 5, the first threaded connector 145 can include a first internal thread connector that is inserted into the top inlet of the enema bulb 110, where the first internal thread connector is connected to an inner side thread of a first internal thread connector 180. The anti-reflux coupler 115 can be connected to the lower inner side of a first external thread connector. A second internal thread connector can be mounted on the upper outer thread of the first external thread connector, and the third inner thread connector can be mounted on the outer thread of the first external thread connector. The upper portion of the third internal thread connector can be fitted with a small nozzle 105 (also referred to as a “syringe pipe”), as shown in FIG. 2, and the second internal thread connector can be mounted with a large nozzle 105 externally, as shown in FIG. 1.

The first sealing ring 155 can be mounted at the junction of an external thread connector and a first internal thread connector, as shown in FIG. 1. The second sealing ring 160 can be mounted at the junction of a first external thread connector and a second internal thread connector. The third sealing ring 170 can be mounted at a junction of a first external thread connector and a third internal thread connector.

Referring again to FIGS. 3 and 4, the water outlet 139 can include a first water outlet 190 and a second water outlet 195 respectively mounted in the concave surfaces of the upper side of both the large nozzle 105 (e.g., FIG. 1) and the small nozzle 105 (e.g., FIG. 2). The small nozzle 105 and the large nozzle 105 can include syringe pipes, and the surface has no joint line. All of the cross-sections and edges and corners can be smooth to avoid friction or sharp edges when inserting into a bodily cavity. The top of the anti-reflux coupler 115 can be mounted with an anti-reflux diaphragm 128. The lower portion of the upper side thread of the anti-reflux coupler 115 can be mounted with a fourth sealing ring 193, and the side of the anti-reflux coupler 115 can be mounted with the water outlet 139.

When the anti-reflux enemator 100 is in operation, the solution is contained in the enema bulb 110. When the solution is squeezed, for instance, to supply the solution, the solution opens the anti-reflux diaphragm 128 on the upper portion of the anti-reflux coupler 115, as shown in the partial, cross-section view of the anti-reflux coupler 115 in FIG. 7, which flows into the nozzle 105 from the water outlet 139 (and the first water outlet 190 and the second water outlet 195) on the top side of the nozzle 105, thereby flowing into the human body.

At the same time, reflux is applied to the solution by a counter force of air pressure in the human body. At this time, the anti-reflux diaphragm 128 is closed by the counter force to prevent the solution from flowing back into the enema bulb 110. After the enema bulb 110 is no longer squeezed, the enema bulb 110 inhales air outside of both the first one-way air valve 125a and the second one-way air valve 125b at the bottom of the enema bulb 110, by elastic rebound for example, and the enema bulb 110 returns to its original shape. Then, the first one-way air valve 125a and the second one-way air valve 125b stop the intake of air, and the first one-way air valve 125a and the second one-way air valve 125b both have an anti-reflux function similar to the anti-reflux coupler 115 and the anti-reflux diaphragm 128, which greatly reduces the risk of the solution leaking. As shown in FIG. 6, the first one-way air valve 125a can be arranged at a middle position of the bottom of the enema bulb 110. The second one-way air valve 125b can be mounted in the first one-way air valve 125a.

In the process of supplying the solution, sealing gaskets are used for sealing in multiple places, and the solution inside the enema bulb 110 is prevented from flowing out. The fourth sealing ring 193 of the anti-reflux coupler 115 is self-sealed with the first external thread connector after the anti-reflux coupler 115 is tightened, so that the reflux solution does not leak from the side. A water outlet hole on the side of the anti-reflux coupler 115 can allow the remaining solution for use to pass without wasting any solution.

In some embodiments, the nozzle 105 (including the small nozzle 105 and/or the large nozzle 105) in this embodiment are formed of polyvinyl chloride (PVC) or have a PVC coating formed thereon. Notably, the surface of the nozzle 105 has no joining line or, in other words, no line where two pieces of the nozzle 105 are mated. The triangular cross-section provides smooth edges and corners, and is convenient for entering the anus, the intestine, or other bodily cavity for cleaning. The first water outlet 190 and the second water outlet 195 are integrally formed on a concave surface of the triangle. As shown in FIGS. 3 and 4, the first water outlet 190 and the second water outlet 195 are not easily blocked during use, as they provide a large water outflow.

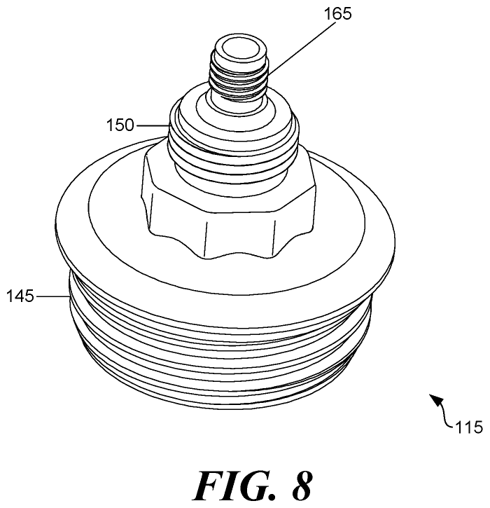

In some embodiments, the large nozzle 105 shown in FIG. 1 is set in a curved shape, which is easier to enter the human body, and is internally mounted with a second internal threaded connector. On the other hand, the small nozzle 105, as shown in FIG. 2, can be in the shape of an elongated cucurbit, and the third internal threaded connector is mounted therein. As such, the anti-reflux enemator 100 is more convenient to disassemble, clean, and reassemble, while also providing different choices for people with different needs. In some embodiments, the first external thread connector can employ a skidproof octagonal plastic structure, which is convenient for hand-tightening and is more convenient to disassemble, as shown in FIG. 8.

The small nozzle 105 (e.g., FIG. 2) and the large nozzle 105 can include dipping pipes, which are formed using a plastic dipping process, and the surface is smooth without a joint line. To this end, in some embodiments, the dipping process can include: (1) mold heating: heating in an oven at 200-250° (or 10 minutes, the first mold heating time needs to be appropriately lengthened, so as to avoid the mold temperature being too high, and the material thickness not being up to standard; the PVC material is heated to approximately 35˜40° C.; (2) dipping: the mold is moved above the trough, the trough is raised to the required height for 3 to 8 minutes, and the dipping length between 120 and 140 mm is performed to avoid the product being scrapped when the cut product is not long enough, or the cut product is too long affecting the total length of the subsequent cut to increase the difficulty of operation; after the dipping is completed, the mold slowly leaves the dipping trough vertically, and is transferred into the oven for baking at the oven temperature of 160˜190° C. for 580˜620 seconds; (3) cooling and demolding: the device is removed from the oven area and placed in a cooling zone for 5 to 10 minutes. The time is determined according to the wall thickness of the product; the demolding operation is performed after proper cooling; (4) head trimming: Any bumps on the nozzle 105 are grinded into a circular arc shape by a polishing wheel, and then baked with a heat gun until the surface is smooth and shiny; (5) Cutting length: If the product is too long, cut the length of the product to 120-140 mm with a pipe cutter, and then use the cutter tool to cut off the excess part of the tail to make the total length reach 115-125 mm; (6) head punching: Three holes are punched in the three planes of the triangular head with tooling and 4-6 mm drill bit; (7) cleaning: Wipe the surface with a dust-free cloth to ensure that each of them is clean; (8) bonding plastic parts: The internal thread plastic parts are bonded to the inside; (9) inspection: inspect whether the appearance and hardness meet the requirements of the customer and whether the head is smooth; and (10) packaging: the nozzle 105 is placed neatly into the packaging carton to prevent deformation due to mutual extrusion.

The features, structures, or characteristics described above may be combined in one or more embodiments in any suitable manner, and the features discussed in the various embodiments are interchangeable, if possible. In the following description, numerous specific details are provided in order to fully understand the embodiments of the present disclosure. However, the person skilled in the art will appreciate that the technical solution of the present disclosure may be practiced without one or more of the specific details, or other methods, components, materials, and the like may be employed. In other instances, well-known structures, materials, or operations are not shown or described in detail to avoid obscuring aspects of the present disclosure.

Although the relative terms such as “on,” “below,” “upper,” and “lower” are used in the specification to describe the relative relationship of one component to another component, these terms are used in this specification for convenience only, for example, as a a direction in an example shown in the drawings. It should be understood that if the device is turned upside down, the “upper” component described above will become a “lower” component. When a structure is “on” another structure, it is possible that the structure is integrally formed on another structure, or that the structure is “directly” disposed on another structure, or that the structure is “indirectly” disposed on the other structure through other structures.

In this specification, the terms such as “a,” “an,” “the,” and “said” are used to indicate the presence of one or more elements and components. The terms “comprise,” “include,” “have,” “contain,” and their variants are used to be open ended, and are meant to include additional elements, components, etc., in addition to the listed elements, components, etc. unless otherwise specified in the appended claims. The terms “first”, “second”, etc. are used only as labels, rather than a limitation for a number of the objects.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, the following is claimed:

1. An enemator, comprising:
a nozzle;
an enema bulb fluidly coupled to the nozzle, the enema bulb being configured to store solution therein and, in response to a squeezing force applied to the enema bulb, direct the solution through the nozzle and a nozzle outlet;
an anti-reflux coupler positioned between the enema bulb and the nozzle through which the solution passes from the enema bulb to the nozzle, the anti-reflux coupler comprising:
an inlet for receiving at least a portion of the solution from an interior of the enema bulb;
an outlet for expelling the solution into the nozzle;
an anti-reverse diaphragm configured to prevent reflux of the solution into the enema bulb from the nozzle; and
a check valve body, the inlet being positioned on the check valve body;
a first threaded connector for detachably attaching the anti-reflux coupler to the enema bulb;
a second threaded connector for detachably attaching the nozzle to the anti-reflux coupler;
a first sealing ring for sealing the anti-reflux coupler and the enema bulb at the first threaded connector;
a second sealing ring for sealing the nozzle and the anti-reflux coupler; and
a first one-way air valve and a second one-way air valve, both being positioned relative to an aperture located at a base of the enema bulb.

2. The enemator of claim 1, wherein the nozzle comprises a triangular-shaped cross-section having a plurality of rounded corners.

3. The enemator of claim 1, wherein:
the nozzle of the enemator is a first nozzle having a first predetermined length; and
the enemator further comprises a second nozzle having a second predetermined length different from the first predetermined length, the second nozzle being configured to replace the first nozzle.

4. The enemator of claim 1, further comprising a third sealing ring for sealing an internal portion of the nozzle to the anti-reflux coupler, the third sealing ring being positioned above the second sealing ring.

5. The enemator of claim 1, further comprising a fourth sealing ring for sealing the check-valve body to a base of the anti-reflux coupler.

6. An enemator, comprising:
a nozzle;
an enema bulb fluidly coupled to the nozzle, the enema bulb being configured to store solution therein and, in response to a squeezing force applied to the enema bulb, direct the solution through the nozzle and a nozzle outlet;
an anti-reflux coupler positioned between the enema bulb and the nozzle through which the solution passes from the enema bulb to the nozzle, the anti-reflux coupler comprising an anti-reverse diaphragm configured to prevent reflux of the solution into the enema bulb from the nozzle; and
at least one one-way air valve positioned relative to an aperture located at a base of the enema bulb.

7. The enemator of claim 6, wherein the anti-reflux coupler further comprises:
an inlet for receiving at least a portion of the solution from an interior of the enema bulb;
an outlet for expelling the solution into the nozzle; and
a check valve body, the inlet being positioned on the check valve body.

8. The enemator of claim 6, wherein the at least one one-way air valve further comprises a first one-way air valve and a second one-way air valve positioned relative to the aperture located at the base of the enema bulb.

9. The enemator of claim 6, further comprising:
a first threaded connector for detachably attaching the anti-reflux coupler to the enema bulb;
a second threaded connector for detachably attaching the nozzle to the anti-reflux coupler;
a first sealing ring for sealing the anti-reflux coupler and the enema bulb at the first threaded connector; and
a second sealing ring for sealing the nozzle and the anti-reflux coupler.

10. The enemator of claim 6, wherein the nozzle comprises a triangular-shaped cross-section having a plurality of rounded corners.

11. The enemator of claim 6, wherein:
the nozzle of the enemator is a first nozzle having a first predetermined length; and
the enemator further comprises a second nozzle having a second predetermined length different from the first predetermined length, the second nozzle being configured to replace the first nozzle.

12. The enemator of claim 8, further comprising a third sealing ring for sealing an internal portion of the nozzle to the anti-reflux coupler, the third sealing ring being positioned above the second sealing ring.

13. The enemator of claim 11, further comprising a fourth sealing ring for sealing the check-valve body to a base of the anti-reflux coupler.

14. A method, comprising:
providing an enemator, the enemator comprising:
a nozzle;
an enema bulb fluidly coupled to the nozzle, the enema bulb being configured to store solution therein and, in response to a squeezing force applied to the enema bulb, direct the solution through the nozzle and a nozzle outlet;
an anti-reflux coupler positioned between the enema bulb and the nozzle through which the solution passes from the enema bulb to the nozzle, the anti-reflux coupler comprising an anti-reverse diaphragm configured to prevent reflux of the solution into the enema bulb from the nozzle; and
at least one one-way air valve positioned relative to an aperture located at a base of the enema bulb.
